(19) United States
(12) Patent Application Publication
(10) Pub. No.: US 2023/0326562 A1
(43) Pub. Date: Oct. 12, 2023

(54) SAMPLE STATUS VISUALIZATION METHODS AND DIAGNOSTIC LABORATORY SYSTEMS INCLUDING SAME

(71) Applicant: Siemens Healthcare Diagnostics Inc., Tarrytown, NY (US)

(72) Inventor: Elizabeth C. Morales, Succasunna, NJ (US)

(73) Assignee: Siemens Healthcare Diagnostics Inc., Tarrytown, NY (US)

(21) Appl. No.: 18/043,995

(22) PCT Filed: Sep. 15, 2021
(86) PCT No.: PCT/US2021/071459

§ 371 Date: Mar. 3, 2023

Related U.S. Application Data
(60) Provisional application No. 63/105,422, filed on Oct. 26, 2020.

(51) Int. Cl. G16H 10/40; G16H 40/20; G06F 3/14; G01N 35/00

(52) U.S. Cl. CPC G16H 10/40; G16H 40/20; G06F 3/14; G01N 35/00871; G16H 40/40

(57) ABSTRACT

Methods of visualizing sample status within a diagnostic laboratory system are provided. The methods include displaying on a display screen, a visual image representing a layout of a plurality of laboratory analyzers included within the diagnostic laboratory system, and further to display on the visual image, for a particular selected sample scheduled for testing, status indicators located proximate to the visual images of the analyzers on which the tests are scheduled. The status indicators denote: the testing is completed on one or more analyzers (processed), the testing is currently being conducted on one or more analyzers (current), or the testing has not yet been received on the one or more analyzers (to do). Systems including such sample status indicators are provided as are other aspects.

### FIELD

This disclosure relates to systems and methods that provide insight into the performance of a diagnostic laboratory system.

### BACKGROUND

Large-scale diagnostic laboratory systems may include a large number of analyzers (e.g., 20 or more) each capable of carrying out a menu of tests on biological specimens, such as whole blood, serum, plasma, urine, cerebral-spinal fluid, interstitial fluid, saliva, and the like. In addition to the analyzers, there may be ancillary test processing equipment such as one or more sample container input/output (I/O) loaders, de-sealers, centrifuges, quality check modules, decappers, aliquoters, and the like that may pre-process the samples and/or sample containers before they arrive at an analyzer for actual testing of the samples.

Many of the analyzers may have test menus that have similar or overlapping capabilities in that they may run a large number of the same or differing tests thereon. The operations of such large-scale diagnostic laboratories undergo continuous monitoring, evaluation, and intervention/manipulation by human operators, which may affect how and where tests are performed on the samples in queue. This may be done to allow for STAT tests to be incorporated, maintenance to be conducted, and to account for occasional unintended shutdowns of one or more analyzers, for example.

However, in some respects existing diagnostic laboratory systems can be deficient.

### SUMMARY

According to a first aspect, a method of identifying status of samples of a diagnostic laboratory system is provided. The method includes providing a visual image of the diagnostic laboratory system including analyzers; and displaying on the visual image proximate analyzers on which a test is scheduled, for a particular selected sample scheduled for testing, status indicators that denote a status of the test at each analyzer having a test scheduled on the particular selected sample.

In a further aspect, a diagnostic laboratory system is provided. The diagnostic laboratory system includes a plurality of analyzers in the diagnostic laboratory system; a process manager server in communication with the plurality of analyzers, the process manager server including data input and a display screen configured and operable, through executable instructions executable on a processor, to: generate and display on the display screen, a visual image of the diagnostic laboratory system including visual images of at least the plurality of analyzers; and display on the visual image, for a particular selected sample scheduled for testing, status indicators located proximate to the visual images of the analyzers on which the tests are scheduled, wherein the status indicators denote: the testing is completed on one or more analyzers, the testing is currently being conducted on one or more analyzers, or the testing is pending on one or more analyzers.

Still other aspects, features, and advantages of this disclosure may be readily apparent from the following description and illustration of a number of example embodiments, including the best mode contemplated for carrying out the disclosure. This disclosure may also be capable of other and different embodiments, and its several details may be modified in various respects, all without departing from the scope of the disclosure. This disclosure is intended to cover all modifications, equivalents, and alternatives falling within the scope of the claims and their equivalents.

### BRIEF DESCRIPTION OF THE DRAWINGS

The drawings, described below, are for illustrative purposes and are not necessarily drawn to scale. Accordingly, the drawings and detailed description are to be regarded as illustrative in nature, and not as restrictive. The drawings are not intended to limit the scope of the disclosure in any way.

FIG. 1A illustrates a schematic diagram of a first diagnostic laboratory system including analyzers, ancillary test processing equipment, and a process manager server that includes a sample status module configured to provide visual status input to a display screen according to one or more embodiments.

FIG. 1B illustrates a display screen having displayed thereon a 2D top view schematic diagram of a diagnostic laboratory system including status indicators provided directly adjacent to respective analyzers to provide status visualization for a particular selected sample according to one or more embodiments.

FIG. 2A illustrates an input box used to input a sample identifier (e.g., sample number) to enable and initiate a search for the status of a particular sample that has (e.g., sample history) or is scheduled for one or more tests on one or more analyzers of the diagnostic laboratory system, according to one or more embodiments.

FIG. 2B illustrates the input box and populated locations below the input box that are populated with one or more date and time stamps for the queried sample, such as collection, order received, sample loaded on an I/O loader of the diagnostic laboratory system, and order completed, according to one or more embodiments.

FIG. 2C illustrates an output box and populated items related to the status of particular tests that were scheduled for a particular analyzer, according to one or more embodiments.

FIG. 2D illustrates an output box and populated items related to the status of particular tests that were scheduled for particular analyzers, according to one or more embodiments.

FIG. 3A illustrates a side view of a sample in a sample container according to one or more embodiments.

FIG. 3B illustrates a side view of a sample container held in a carrier residing on a track according to one or more embodiments.

FIG. 4 illustrates a flowchart of a method of identifying status of samples of a diagnostic laboratory system according to one or more embodiments.

### DETAILED DESCRIPTION

In some instances, it may be desirable to be able to review the status of testing of a particular sample that has been scheduled for testing or that has already been tested in a diagnostic laboratory system. For example, it may be desirable to check the status of a sample that is a STAT sample, or a sample that a healthcare provider has specifically requested the status of, or a routine sample, for whatever reason, a status thereof is of concern. The present disclosure is directed status visualization systems and methods. The status visualization systems and methods described herein allow a user to query a status of a particular sample that has been scheduled for testing within a diagnostic laboratory system. In particular, the status visualization systems and methods provide a visual status indicator that is located adjacent to an image of the one or more analyzers within a visual image of the diagnostic laboratory system that has an ordered test. Thus, the user can immediately receive visual feedback of sample status. The status indicators may be color coded, shaded, hatched, or otherwise differentiated to provide various status information, such as “processed,” “current,” or “to do.” Other statuses may be coded, such as cancelled.

In cases of workload disruptions or changes, workload may be shifted to other analyzers within the diagnostic laboratory system. In such instances, the systems and methods have the ability to rapidly understand and visualize how any such changes made have affected any particular sample's status within the diagnostic laboratory system. Additionally, a location of the sample in the system can be visualized as well.

Therefore, embodiments of the systems and methods herein may advantageously provide improved visualization of any sample's status within the diagnostic laboratory system and, in particular, can provide improved visualization of any queried sample status. Providing improved ability to visualize sample status can also enable any sample to be found and retrieved from the diagnostic laboratory system, if desired. Thus, the ability to rapidly visualize sample status can lead to improved system flexibility.

Thus, in accordance with this disclosure, embodiments provide methods and systems that provide improved sample status visualization within a diagnostic laboratory system. In some embodiments, the systems and methods providing such improved sample status visualization may be provided by utilizing color as one means to visualize status. The color may be used as part of a dynamically-changeable color status indicator in, or otherwise disposed on, a visual image of the laboratory system. In particular, colors can be assigned and such individual colors can be associated with a particular status, such as: 1) the testing a sample is completed on one or more of the analyzers (processed), 2) the testing on a sample is currently being conducted on one or more analyzers (current), and/or 3) the testing is not yet pending on one or more analyzers (to do).

In some embodiments, the status indicators can also provide a status of the sample relative to one or pieces of ancillary test processing equipment that are included in the diagnostic laboratory system and displayed on the display screen. In some embodiments, the individual color overlays can overlie a 2D or even 3D graphical image of the analyzers and/or pieces of ancillary test processing equipment that are displayed on the display screen. Individual color overlays are color changeable to signify a change in status.

A color (e.g., green, yellow, red, etc.) may be assigned to each status to be visualized. The status data can be received from any suitable source, such as from a middleware server 105, which can communicate with each of the analyzers and/or each of the pieces of ancillary processing equipment, for example. Communication may be through a local area network (LAN), wide area network (WAN), or other suitable wired or wireless communication system. For example, the instantaneous status data may be received from a middleware database, which has stored the status data as the various analyzers report their status updates. Such status data may be provided to the middleware server by any suitable means, such as an output signal from an analyzer and/or each piece of ancillary processing equipment that can provide sample ID and sample status data concerning the status of any samples that are being processed or tested or have completed processing or testing.

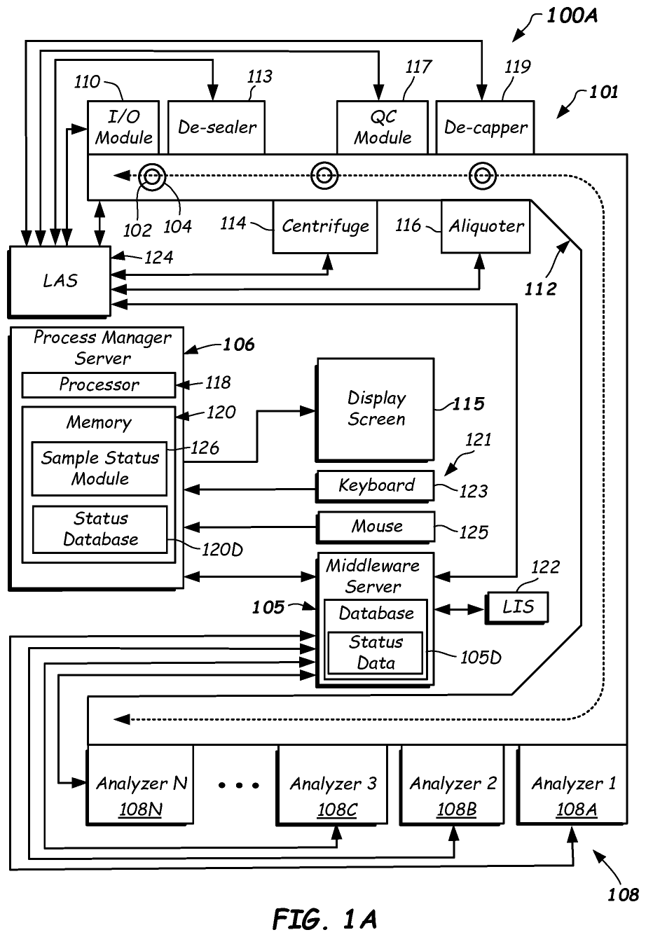

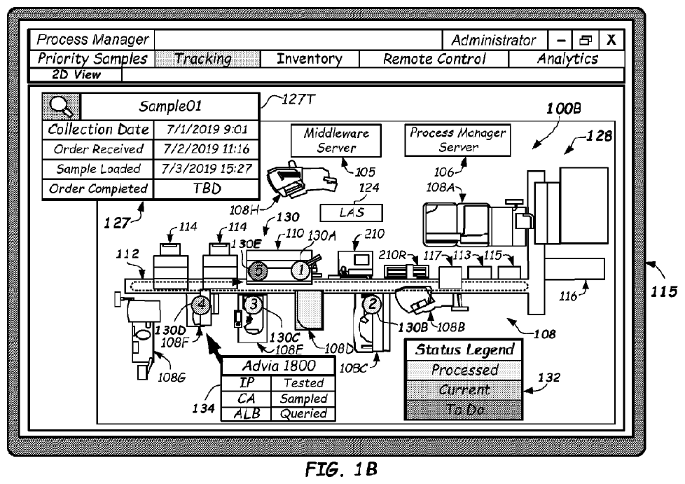

Further details of inventive diagnostic laboratory system and methods will be described with reference to FIGS. 1A-4 herein.

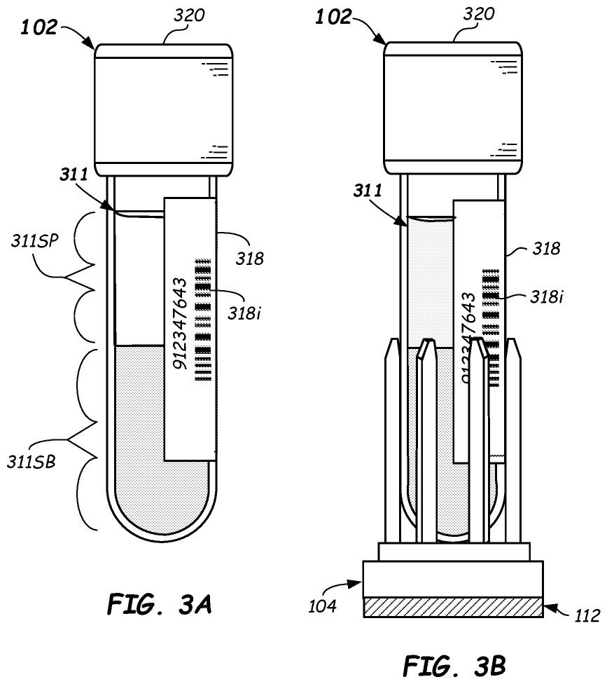

FIG. 1A illustrates a diagnostic laboratory system 100 according to embodiments of the disclosure. Diagnostic laboratory system 100 may automatically process large numbers of biological samples 311 (FIGS. 3A-3B) aspirated from sample containers (e.g., sample containers 102), which can be transported on carriers 104 to a number of analyzers 108 and ancillary test processing equipment 101 with minimal human intervention, except possibly for the introduction of STAT tests, maintenance, and service for breakdowns and work stoppages. Diagnostic laboratory system 100 may include a process manager server 106 and a plurality of analyzers 108 (represented specifically by laboratory analyzers 108A, 108B, and 108C-108N). Any number of analyzers 108 may be shown.

Diagnostic laboratory system 100 may further include one or more pieces of ancillary test processing equipment 101, such as, for example, a input/output (I/O) module 110 that is configured to load and/or unload sample containers 102 from a track 112, de-sealer 113 that is configured to unseal a cap 320 of the sample container 102, a centrifuge 114 configured to settle red blood cells from whole blood, a de-capper 119 that is configured to remove the cap 320 from the sample container 102, and an aliquoter 116 that is configured to aspirate and dispense a small amount of the sample 311 into another container, which then can be forwarded to an analyzer 108 for testing. Other ancillary test processing equipment 101 may be included, such as a quality check (QC) module 117, configured to check the sample 311 or the sample container 102 for one or more characteristics, such as a volume of serum or plasma, the presence of an interferent such as Hemolysis, Icterus, Lipemia (HIL) in the serum or plasma, or the presence of an artifact such as a blood clot, bubble, or foam therein, the size of the sample container 102, or the like. Ancillary test processing equipment 101 as used herein is any piece of equipment that is ancillary to the analyzers 108A-108N in that the piece of ancillary test processing equipment 101 performs a “process, test, or characterization” (hereinafter pre-processing) on the sample 311 or on the sample container 102 prior to analysis is of the sample 311 by one or more of the laboratory analyzers (108A, 108B, and 108C-108N).

The track 112 may be configured to transport sample containers 102 containing samples 311 (FIG. 3B) in carriers 104 to and from the I/O Module 110 as well as to and from other ones of the ancillary test processing equipment 101 and to and from each of the analyzers 108A-108N within diagnostic laboratory system 100. As shown in FIGS. 3A and 3B, sample containers 102 may comprise tubular containers each be provided with one or more labels 318 that may include information 318i thereon, such as, a time and/or date stamp, requested test(s), patient identification, accession number, and the like. The label(s) 318 may include, e.g., a barcode and/or have alphanumeric information printed thereon. The barcode and/or identification information may be machine readable at various locations about diagnostic laboratory system 100 so that arrival of a particular sample 311 at any location within the diagnostic laboratory system 100 may be determined. Sample containers 102 may include caps 320 and may be sealed to the tube in some instances. Track 112 may be a railed track (e.g., a mono rail or a multiple rail), a collection of conveyor belts, conveyor chains, moveable platforms, or any other suitable type of conveyance mechanism. Some embodiments of the track 112 may be U-shaped as shown, circular, or other suitable shapes and may be a closed track (e.g., an endless track), and may have paths as offshoots from a main track in some embodiments. Carriers 104 may move both ways on the track 112. In some embodiments, the carriers 104 can include a linear motor vehicle that is programmed to stop at the desired locations including at ancillary test processing equipment 101 and at one or more of the analyzers 108 that have tests scheduled thereon. In some embodiments, the sample 311 is a fractionated (centrifuged) sample of whole blood, wherein a serum or plasma portion 311SP is separated from a settled blood portion 311SB. In many tests, a small amount of the serum or plasma portion 311SP can be aspirated by an analyzer 108 and testing is conducted on the sample 311. While the testing is being completed at an analyzer 108, the sample 311 in sample container 102 may progress onward to another analyzer or I/O module 110.

Again referring to FIG. 1A, a laboratory automation system (LAS) 124, which can be a server or other suitable computer or processor, can be provided to control the track 112 and the movement of the various samples 311 within the diagnostic laboratory system 100. For example, middleware server 105 can be configured to receive test orders for the various samples 311 from a laboratory information system LIS 122, which, in turn, may have received test orders from a hospital information system (not shown), which may be input by a doctor or other health professional. Thus, when a sample 311 is loaded onto an I/O module 110, information 318i is read (e.g., via reading a barcode) which then queries the middleware server 105 to receive information on the tests that are, or have been, scheduled for that particular sample 311. Such information on the tests to be run on a particular sample 311 can be stored as status data 105D in a database of the middleware server 105. Thus, database of the middleware server 105 can hold status data 105D on the tests ordered on samples 311, which may be tied to an accession number or other like identifier. Based on the samples 311 that are known to have been received in the diagnostic laboratory system 100, a plan can be generated for the samples in queue for processing in terms of which analyzers 108 will be used, and in what sequence they will be used to carry out the test orders. Any suitable means for determining the testing plans for samples in queue may be used. Such plans may be generated by middleware server 105, or optionally, by LAS 124, according to any suitable logic. In some embodiments, the processing of which analyzers 108 will be used, and in what sequence the tests are to be run can be generated after a load plan, such as an optimized load plan, has been generated for setup of the test menus and reagents that are to be assigned on each of the analyzers 108.

As shown, the diagnostic laboratory system 100 further includes a process manager server 106. Process manager server 106 can be provided in digital communication with middleware server 105 over a LAN or other suitable communication system, for example. Process manager server 106 may include processor 118, e.g., a microprocessor-based, central processing unit (CPU) or other suitable digital processor configured to execute programming instructions, memory 120, such as a combination of random access memory (RAM) and read-only memory (ROM), to store sample status software, firmware, and other suitable electronics and hardware configured for communication with the middleware server 105. Process manager server 106 can further include input peripherals 121, such as keyboard 123 and mouse 125, and an output peripheral such as a display screen 115. Memory 120 can store programming instructions executable by the processor 118 enabling the display of status information generated by sample status module 126. The output peripheral (e.g., display screen 115) is configured to display status information about the status of any queried sample 311 that is, or was, scheduled for a test on the various analyzers 108A-108N and/or scheduled for pre-processing on one or more pieces of ancillary test processing equipment 101 of the diagnostic laboratory system 100. Each of the analyzers 108A-108N is configured to perform one or more types of diagnostic tests and/or analyses on samples 311 delivered thereto by the track 112 or otherwise.

The diagnostic testing carried out on the analyzers 108A-108N can include, but is not limited to, immunoassay testing (e.g., chemiluminescent immunoassays (CLIA), radioimmunoassays (RIA), counting immunoassays (CIA), fluoroimmunoassays (FIA), and enzyme immunoassays (EIA and including enzyme linked immunosorbent assays (ELISA)), to target a specific target biomolecule, clinical chemistry analyzers to measure concentrations of substances (e.g., glucose, Hemoglobin A1C, lipids (fats), triglycerides, blood gases (e.g., carbon dioxide, etc.), enzymes, electrolytes (e.g., sodium, potassium, chloride, and bicarbonate), lipase, bilirubin, creatinine, blood urea nitrogen (BUN), hormones (e.g., thyroid stimulating hormone), hepatitis, minerals (e.g., iron. calcium, magnesium, etc.), proteins, and other metabolic products and the like) in samples 311. Other chemical, assay, protein, or other constituent testing may be performed on the samples 311 by the analyzers 108A-108N of diagnostic laboratory system 100.

In some embodiments, many of the analyzers 108A-108N of the diagnostic laboratory system 100 may be capable of performing the same menu of tests, while others of the analyzers 108A-108N may be capable of performing only a limited number of tests or only certain individual tests. There may be test menu overlap in some cases. In some embodiments, diagnostic laboratory system 100 may have 20 or more, 30 or more, 40 or more, 50 or more, 60 or more, 70 or more, 80 or more, 90 or more, or even 100 or more analyzers 108A-108N (where N can be any integer). Each of the analyzers 108A-108N can consume inventory and an amount of inventory present on one or more of the analyzers 108A-108N (e.g., a numerical count of an amount of reagent (e.g., volume), number of substrates, number of containers, and the like) can be communicated to the middleware server 105 from each analyzer 108.

Further, status information concerning the samples 311 scheduled for, undergoing, or having completed testing at one or more analyzers 108 may be recorded by middleware server 105 and stored in database as status data 105D thereof. Particular status information of one or more samples 311 may be communicated to the process manager server 106 as requested via a query input via input peripherals 121. Optionally, all status information may update in real time and can be stored in a status database 120D in memory 120 of the process manager server 106, such as for redundancy and ready and unfettered access to the data thereof.

For example, as a particular sample test plan is generated by LAS 124 for the samples 311 that are available in the diagnostic laboratory system 100, each sample 311 may receive a status that the testing is not yet received on one or more of the analyzers 108 (i.e., given a “to do” status). Thus, for example, if no testing has yet commenced, i.e., the samples are still either on track 112 or still at I/O module 110, then a “to do” status is assigned to the tests scheduled for that particular sample 311, provided they have not yet been analyzed at any analyzer 108. Status information may also include the location of a particular queried sample if the sample is located at a piece of ancillary test processing equipment 101.

As a sample 311 scheduled for a particular test or tests to be conducted at an analyzer (e.g., analyzer 108A-108N) is received at a particular analyzer 108 by being transported on track 112 under the control of the LAS 124, the information 318i may be read on sample container 102 by a reader device (e.g., barcode reader) or the like at the analyzer 108. LAS 124 may then be queried as to which test or tests to run at the particular analyzer 108. When the particular analyzer 108 has received the sample 311 and now knows what test or tests to run on the particular sample 311, then the status may be toggled to “current” status, and the new status stored as status data 105D in the database of the middleware server 105 and/or status database 102D. “Current status” means that the sample 311 has arrived at the analyzer, and a test or tests (testing) is currently in queue or actually being conducted on the particular analyzer 108.

When the testing is completed on the particular analyzer 108 and the middleware server 105 determines that the results make sense, such as by being within a pre-defined range and that applicable QC testing was passed, for example, the status of testing on that sample 311 at that particular analyzer 108 may be toggled to “processed” status and the new status stored as status data 105D in the database of the middleware server 105 and/or status database 120D. Thus, the status can remain in current status until all the testing at that analyzer 108 for that sample 311 is completed. If multiple tests are ordered for that analyzer 108, then each analyzer may have its own status updates. Such status updates will continue as the samples 311 progress through the diagnostic laboratory system 100 so that for all samples 311 in the diagnostic laboratory system 100, a status designation of “to do”, “current” or “processed” exists for all analyzers 108 that had a scheduled test thereon. Thus, for any particular sample 311, depending upon how much of the testing has been completed; the status may include any combination of status designations of “to do”, “current” or “processed.”

Additionally, as the sample 311 progresses to, and is pre-processed, by any of the pieces of ancillary test processing equipment 101, a status of the sample 311 at the piece of ancillary test processing equipment 101 may also be toggled between “to do,” “current,” or “processed.” In this instance, “to do” status means that pre-processing is scheduled, but that the sample 311 has not yet arrived at the specific piece of ancillary test processing equipment 101. Current means that the sample 311 has arrived at the piece of ancillary test processing equipment 101, as determined by information 318i being read, and that the process is in queue or is currently being performed. Upon being read, the new status can be stored as status data 105D in the database of the middleware server 105 and/or in status database 120D. When the pre-processing is completed on the specific piece of ancillary test processing equipment 101, the status of pre-processing on that sample at that particular analyzer may be toggled to “processed” status and the new status stored in the database 105D and/or in status database 120D. Middleware server 105 may contain middleware in memory, which is a software program that assists with inventory and load control of the analyzers 108A-108N and/or pieces of ancillary test processing equipment 101.

Referring now to FIG. 1B, a display screen 115 is shown that may be communicatively coupled to the process manager server 106, such as by a communication cable and suitable graphics card or other suitable connection. Display screen 115 may be included in any suitable display component or apparatus including, for example, a display frame and display screen 115. Display screen 115 may comprise a cathode ray tube (CRT) display, a light-emitting diode (LED) display, an electroluminescent display (ELD), electronic paper or E ink, a plasma display panel (PDP), a liquid crystal display (LCD), an organic light-emitting diode (OLED) display, a digital light processing (DLP) display, and the like, that is configured to display a visual image of the layout. Display screen 115 can be a touch screen in some embodiments, Display screen 115 can be included in a hand-held device, such as a cell phone or a tablet, for example.

Thus, it should be recognized that status updates can be provided in real time and may be updated over a relatively short period of time or instantaneously. In some embodiments, the display of status can be provided as a dynamically-changeable overlay on the display screen 115. The particular status data to be displayed can be selected by the user (is user selectable) such as by a suitable sample ID selection method. For example, the sample may be queried by typing a sample ID (e.g., Sample 01) into a search field, such as in a text box 127T displayed on the display screen 115.

As shown in FIG. 1B, the display screen 115 is configured to display an image 128 of a layout of the diagnostic laboratory system 100. For example, the image 128 of the layout may be a two-dimensional (2D) image, such as a top plan image as shown, or optionally a three-dimensional (3D) image, each of which showing the relative or approximate locations of the analyzers 108A-108N within the diagnostic laboratory system 100. The display screen 115 may also be configured to display in the image 128, a relative or approximate location of the one or more pieces of ancillary test processing equipment 101 in the diagnostic laboratory system 100. For example, the pieces of ancillary test processing equipment 101 can be one or more I/O devices, such as I/O module 110, bulk I/O device 210, rack I/O device 210R, one or more centrifuges 114, one or more QC modules 117, one or more de-sealers 113, one or more de-cappers 119, QC module 117, and/or aliquoter 116, for example. Some of the functions of the afore-mentioned may be consolidated in one device located along the track 212. The ancillary test processing equipment 101 can be arranged around the track 212 in any efficient manner. In some embodiments, even the track 212 can be considered a piece of ancillary test processing equipment 101. Other or different types of ancillary test processing equipment 101 can be displayed as part of the image 128.

Again referring to FIGS. 1A and 1B, overlaying the image 128 of the layout of the analyzers 108A-108N including the layout of one or more pieces of ancillary test processing equipment 101 in the diagnostic laboratory system 100 are one or more dynamically-changeable status indicators 130 disposed on the image 128 on the display screen 115. For example, the one or more dynamically-changeable status indicators 130 may be displaying on the image 128 proximate analyzers 108 and of the ancillary test processing equipment 101, if used. The status indicators 130 may be disposed and positioned such that they are un-mistakenly associated with one or the analyzers 108 and of the ancillary test processing equipment 101, if used thereon. This each status indicator 130 can be associated with an analyzer 108 or a piece of ancillary test processing equipment 101. The image 128 may include an actual outline or representation of the actual analyzers 108 and/or pieces of ancillary test processing equipment 101 in 2D, such as shown in FIG. 1B. Optionally, as shown in FIG. 1A, the representation of the analyzers 108 and/or pieces of ancillary test processing equipment 101 that is displayed on the display screen 115 may be simply boxes or other polygonal elements arranged to approximate the arrangement in the diagnostic laboratory system 100A. In some embodiments, status indicators 130 can be accompanied on the image 128 with an image of a process path (dotted line possibly including arrows indicating path direction) of the sample 311.

One possible embodiment of one or more of the dynamically-changeable status indicators 130 can be a geometric article, such as a circle, square, rectangle, triangle, hex, or the like. Any desired polygonal shape may be used. The geometric article can include a sequence indicator associated therewith, such as a number (shown) or possibly a letter, located inside of the geometric article. One example is the use of consecutive numerals (consecutive numbers 1 through 5 as shown in FIG. 1B) located inside of a geometric article comprising a circle. The numbers denote the sequence of the various pre-processing at the one or more of the ancillary test processing equipment 101, if used, and/or the sequence of the tests taking place at various analyzers 108.

As shown, the dynamically-changeable status indicators 130 is made up of a plurality of individual status indicators 130A-130E, with each designated with a color that indicates an associated status. The status, can include “to do,” “current,” and “processed” as described herein. In the depicted example, the individual dynamically-changeable status indicators 130A-130E are shown associated with ancillary test processing equipment 101 and analyzers 108. The sample (Sample01) that has been queried at text block 127T has a status of “green” shown at status indicator 130A labeled as 1. That means Sample01 was “processed” by first being input at I/O module 110 and then it has been loaded onto the track 112. Next Sample01 traveled on the track 112 to a next destination at analyzer 108C having status indicator 130B labeled 2 disposed therewith. The status indicator 130B is colored green and numbered as 2, meaning that the testing that had been ordered to be conducted at analyzer 108C by LAS 124 has been “processed” and was released back onto the track 112. The colors associated with each status can be understood by including on the display screen 115, such as adjacent the image 128, a status legend block 132. Any suitable type of status legend block 132 may be used to signify what each of the colors stand for. As shown, green stands for “processed,” yellow stands for “current,” and grey stands for “to do.”

Following on, it can be seen that the testing for Sample01 has been completed at analyzer 108E by illustrating status indicator 130C colored green and labeled 3 located proximate the representative image graphic of the analyzer 108E. Next, status indicator 130D labeled 4 is shown as yellow meaning that testing on analyzer 108F is currently in queue or is being tested. It can further be understood and readily communicated to the viewer that once the testing is completed at analyzer 108F, then Sample01 is scheduled to be unloaded at I/O module 110, as indicated by status indicator 130E labeled 5 and colored grey indicating that the unloading has not yet occurred, i.e., it is in a “to do” status. As shown, each of the status indicators 130 has a border (e.g., circular border shown). However, no such border is needed. For example, a borderless colored geometric shape could be used, with just a numeral (e.g., 1−n, where n is an integer) provided thereon. Further a fuzzy edged color overlay may be used.

The plurality of individual color status indicators 130 as shown (e.g., status indicators 130A-130E) are positioned on the image 128 on the display screen 115 so that they are unambiguously associated with (e.g., at least partially overlap with or unambiguously next to) the respective representative geometric figures in the image 128 representing the analyzers 108A-108N, and the one or more pieces of ancillary test processing equipment 101, is status indicators are used therefor. Thus, associated, as used herein, means that the status indicator 130 is laying on, above, below, surrounding, or immediately proximate the location of the representative geometric figures of the analyzers 108, and the one or more pieces of ancillary test processing equipment 101 (if status indicators are used therefor). A circumscribed area of the individual status indicators 130 may be immediately proximate with, coincide with, partially coincide with, or surround the area of the geometric figures representing the analyzers 108A-108N (and one or more pieces of ancillary test processing equipment 101, if included).

In some embodiments, the geometric shapes representing the analyzers 108A-108N and one or more pieces of ancillary test processing equipment 101 may be completely filled in with color and a sequence indicator (numeral 1−n) may be associated therewith, such as being on or directly next to the analyzers 108A-108N and one or more pieces of ancillary test processing equipment 101. Thus, as should be apparent, many forms of the individual status indicators 130 are possible, such as solid or transparent color, fully overlapping or partially overlapping, fully filling or partially filling, fully or partially surrounding, or immediately proximate. Other suitable methods for denoting the different statuses may be used, such as extent of shading, degree of filling, and using different hatch patterns.

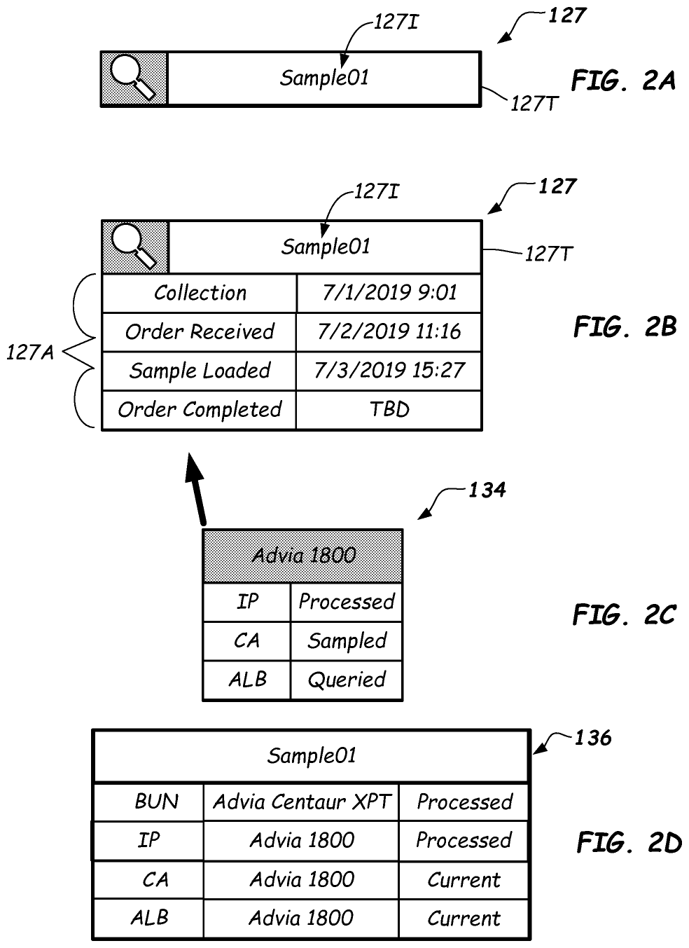

Various embodiments showing different types of menus and text blocks are shown in FIGS. 2A-2D. For example, FIG. 2A illustrates the text block 127 used to allow entry of a sample identifier 1271 (e.g., sample number) whose testing and/or pre-processing status is desired to check. Upon entry of the sample identifier 1271, the additional text boxes 127A may be filled from middleware server database or status database 120D. For example, the additional text boxes 127A may include one or more of the following: collection date and time, when the order was received, when the sample was loaded on an I/O device, and when the testing order for the sample was completed (i.e., last test on an analyzer is completed).

FIG. 2C illustrates a populated box 134 that can be a pop-up or other suitable format. In particular, the populated box 134 can provide, such as when clicking on a particular status indicator (e.g., status indicator 130D) a listing of the particular tests that have been ordered and assigned to that analyzer 108F for that sample (e.g., Sample 01). Thus, in the depicted example, the test type can be populated (e.g., IP, CA, and ALB) standing for agarose gel immunoprecipitation (IP), blood calcium (CA), and albumin (ALB), respectively. Also displayed proximate to the analyzer 108 currently conducting one or more tests on the sample 311 may be the analyzer name (e.g., Advia 1800), which can be an analyzer name, model number, or other given name. Further, the respective status of each test scheduled for that analyzer 140A can be displayed, such as “processed,” when testing is completed and approved. Further, the status, may be more refined than indicating a current status, such as indicating that the sample has been “sampled” or that the sample has been queried, such as by querying the LAS 124 after reading the barcode on the label 318.

FIG. 2D illustrates another option for displaying status in addition to the status indicators 130. Test block 136 can include a description, such as a name of all the tests ordered for the sample, the name of the particular analyzer 108 that is conducting the tests, and the status, in terms of processed, current, or to do.

As should be apparent from the above examples, each of the plurality of individual status indicators 130 can be color changeable, such as between a plurality of colors. For example, the color of each of the plurality of individual status indicators 130 can be changeable individually between two or more colors, three or more colors, four or more colors, or even five or more colors should more gradations of status be desired. Overlaying the representative geometric figures of the analyzers 108 and ancillary test processing equipment 101 in the image 128 with individual color status indicators 130 is used to indicate a particular status of a queried sample for the plurality of analyzers 108A-108N (and optionally also the pieces of ancillary test processing equipment 101 if desired) that are or were scheduled to undergo testing and possibly pre-processing. Thus, at a glance, the user/viewer can readily understand the status of the sample, where it is currently, and what tests remain to be conducted.

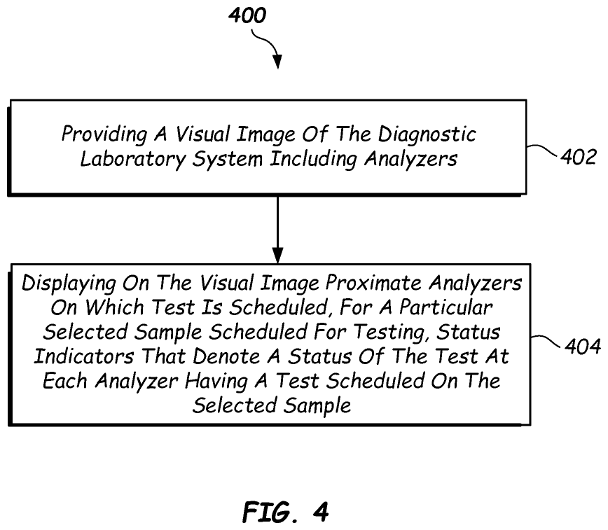

FIG. 4 illustrates a flowchart of a method 400 of identifying status of samples of a diagnostic laboratory system (e.g., diagnostic laboratory system 100A or 100B) according to one or more embodiments of the disclosure. Method 400 may be carried out by any suitable server, such as process manager server 106 and display screen (e.g., display screen 115). Method 400 may include, at process block 402, providing a visual image (e.g., image 128) of the diagnostic laboratory system (100A, 100B) including analyzers (108). The method 400, further includes, in block 404, displaying on the visual image (e.g., image 128) proximate analyzers (e.g., analyzers 108) on which test is scheduled, for a particular selected sample (e.g., sample 311) scheduled for testing, status indicators (e.g., status indicators 130) that denote a status of the test at each analyzer (e.g., analyzer 108) having a test scheduled on the selected sample (e.g., sample 311). See FIG. 1B for an image 128 displayed on a display screen 115. The image 128 and the status indicators 130 may be displayed as pixels on a suitable display screen, such as a computer monitor. Optionally, the image 128 and the status indicators 130 may be displayed as a projected image on a projection surface, wherein the projection surface is the display screen, for example. Other means for displaying the image on a display screen may be used. The image 128 may be generated using any suitable modeling software. The method 400 of identifying status of samples 311 in the diagnostic laboratory system 100A, 100B can be selectable by a user by selecting a tracking tab, as shown highlighted in FIG. 1B.

The displayed image 128 may include any combination of geometric figures representative of the layout of the plurality of analyzers 108A-108N in the diagnostic laboratory system. For example, as best shown in FIG. 1B, ancillary test processing equipment 101 that are devices that perform one or more processes (i.e., pre-processing) on the sample 311 or the sample container 102 prior to being sent to one or more of the analyzers 108A-108N for analysis.

In another embodiment, a non-transitory computer readable storage medium is provided. The non-transitory computer readable storage medium refers to computer-readable media (CRM) that stores data for short periods or in the presence of power such as a memory device or Random Access Memory (RAM). Non-transitory computer readable storage medium further comprises a sample status module 126 having computer-executable instructions (software instructions) that, when executed by the processor 118, cause the processor 118 of the process manager server 106 to perform functions of receiving, storing, generating, and causing display of the image 128 and of the status indicators 130. In particular, the computer-executable instructions enable the receiving of status data from status database 120D or from for the diagnostic laboratory system 100A, 100B comprising a plurality of analyzers 108A-108N. The receiving of status data in the status database 120D can be enabled by a transmission control protocol (TCP) communication protocol or the like.

Further, the computer-executable instructions enable the storage of the status data 105D in a status database 120D. Status data can be data on the statuses of various samples 311 undergoing pre-processing and/or testing in the diagnostic laboratory system 100A, 100B. The status data can be stored in the status database 120D by storage procedures, database writes, or other typical techniques used to store data in a database. Additionally, the computer-executable instructions enable the generation of image data of the image 158 of a layout (the spatial payout) of the plurality of analyzers 108A-108N and layout of the ancillary test processing equipment 101. The image data is generated by determining the status for that queried sample 311 in the analyzer 108A-108N and/or particular piece of ancillary test processing equipment 101 and thereby deriving a color or other feature indicative of that status. The color or other feature indicative is rendered as a dynamically-changeable status indicator 130 for each of the particular analyzer 108A-108N and/or particular piece of ancillary test processing equipment 101 on the display screen 115 that is scheduled to be visited by the sample 311.

Also, the computer-executable instructions enable the generation of the dynamically-changeable status indicators 130. The dynamically-changeable status indicators 130 are generated in software by a graphics generator including a shader with pixel addressability. The colors assigned to each of the status indicators 130 are generated based on the applicable status for the sample at that location. Faceted or linear shading may be used. Optionally ray tracing or Phong shading may be used.

Finally, the computer-executable instructions cause the display of the dynamically-changeable status indicators 130 relative to image 128 of the layout of the plurality of analyzers 108A-108N and ancillary test processing equipment 101 based on the status data, wherein respective indicators (e.g., colors, hatching, or the like) of the dynamically-changeable status indicators are changeable in response to a change in status. The computer-executable instructions generate a scene file that is displayed as of the dynamically-changeable status indicator 130 on the display screen 115. A scene file contains geometry, viewpoint, texture, lighting, and shading information as a description of the virtual scene.

While the disclosure is susceptible to various modifications and alternative forms, specific method and apparatus embodiments have been shown by way of example in the drawings and are described in detail herein. It should be understood, however, that the particular methods and apparatus disclosed herein are not intended to limit the disclosure but, to the contrary, to cover all modifications, equivalents, and alternatives falling within the scope of the claims.

What is claimed is:

1. A method of identifying status of samples of a diagnostic laboratory system, comprising:
providing a visual image of the diagnostic laboratory system including analyzers; and
displaying on the visual image proximate the analyzers on which test is scheduled, for a particular selected sample scheduled for testing, status indicators that denote a status of the test at each analyzer having a test scheduled on the particular selected sample.

2. The method of claim 1, wherein the status indicators comprise at least one of:
a) the testing is completed on one or more of the analyzers (processed),
b) the testing is currently being conducted on one or more analyzers (current), or
c) the testing is pending on one or more analyzers (to do).

3. The method of claim 2, comprising any combination of a) through c).

4. The method of claim 1, comprising receiving data regarding the status of the particular selected sample from middleware.

5. The method of claim 1, wherein the visual image of the diagnostic laboratory system further comprises a layout of one or more ancillary test processing apparatus that are ancillary to the analyzers.

6. The method of claim 1, wherein the visual image of the diagnostic laboratory system is displayed on a display screen.

7. The method of claim 1, comprising a search field on a display screen configured to enter a sample number for the particular selected sample.

8. The method of claim 7, comprising displaying for the particular selected sample, one or more of:
a collection date and time,
an order received date and time, and
a sample loaded date and time.

9. The method of claim 1, comprising displaying a name of an analyzer proximate to an analyzer currently conducting one or more tests on the particular selected sample.

10. The method of claim 9, further comprising displaying a name of a test being conducted on the particular selected sample at a location proximate to the name of the analyzer.

11. The method of claim 1, wherein the status indicators include consecutive numbers illustrating a sequence of the testing scheduled for the particular selected sample.

12. The method of claim 1, wherein the status indicators comprise numbered circles.

13. The method of claim 1, wherein the status indicators comprise consecutive numbers denoting an order of the testing.

14. The method of claim 1, wherein the status indicators comprise coloring that denotes status.

15. The method of claim 14, wherein status corresponding to the coloring is identified in a status legend block.

16. The method of claim 1, wherein the status indicators are accompanied on the visual image with an image of a process path of the particular selected sample.

17. The method of claim 1, wherein the method of identifying status of samples in a diagnostic laboratory system is selectable by a user by selecting a tracking tab.

18. The method of claim 1, further comprising a fillable search field configured to allow input of a sample ID by a user.

19. A diagnostic laboratory system, comprising:
a plurality of analyzers in the diagnostic laboratory system;
a process manager server in communication with the plurality of analyzers, the process manager server including data input and a display screen configured and operable, through executable instructions executable on a processor, to:
generate and display on the display screen, a visual image of the diagnostic laboratory system including visual images of at least the plurality of analyzers; and
display on the visual image, for a particular selected sample scheduled for testing, status indicators located proximate to the visual images of the analyzers on which tests are scheduled, wherein the status indicators denote:
a) the testing is completed on one or more analyzers (processed),
b) the testing is currently being conducted on one or more analyzers (current), or
c) the testing is pending on one or more analyzers (to do).

20. A diagnostic laboratory system of claim 19, wherein the status indicators located proximate to the plurality of analyzers on which the tests are scheduled denote any combination of a) through c).

21. The diagnostic laboratory system of claim 19, wherein the visual image of the diagnostic laboratory system further comprises images of one or more ancillary test processing apparatus that are ancillary to the plurality of analyzers.
